(19) United States
(12) Patent Application Publication
(10) Pub. No.: US 2024/0415673 A1
(43) Pub. Date: Dec. 19, 2024

(54) SHOULDER PATIENT SPECIFIC INSTRUMENT

(71) Applicant: Tornier SAS, Saint Martin (FR)

(72) Inventors: Pierric Deransart, Saint Martin d'uriage (FR); Emmanuel Francois Marie Lizee, Saint Ismier (FR); Delphine Claire Michelle Henry, Saint Ismier (FR); Jean Chaoui, Locmaria-Plouzane (FR); Gilles Walch, Lyon (FR); Pascal Boileau, Nice (FR)

(73) Assignee: Tornier SAS, Saint Martin (FR)

(21) Appl. No.: 18/780,692

(22) Filed: Jul. 23, 2024

Related U.S. Application Data
(62) Division of application No. 17/451,499, filed on Oct. 20, 2021, now Pat. No. 12,097,129.
Application No. 17/451,499 is a division of application No. 16/521,466, filed on Jul. 24, 2019, now Pat. No. 11,179,249.
Application No. 16/521,466 is a division of application No. 15/024,747, filed on Mar. 24, 2016, now Pat. No. 10,405,993.
(60) Provisional application No. 61/903,814, filed on Nov. 13, 2013.

(51) Int. Cl. A61F 2/46; A61B 17/17; A61B 17/56; A61F 2/40

(52) U.S. Cl. CPC A61F 2/4612; A61B 17/1739; A61B 17/1778; A61B 2017/568; A61F 2/4081; A61F 2/4603; A61F 2002/4677

(57) ABSTRACT

A system for guiding a glenoid prosthesis is disclosed. The system includes a glenoid guide that includes: a guide feature; at least three arms having a first end coupled with the guide feature and a second end disposed away from the guide feature; at least three peripheral pegs, each peripheral peg extending from the second end of a corresponding arm and includes an engagement surface configured to engage the scapula of the patient; and a strut extending laterally from the guide feature.

### CROSS-REFERENCE TO RELATED APPLICATIONS

The present application is a divisional of co-pending U.S. patent application Ser. No. 17/451,499, filed Oct. 20, 2021, which is a divisional of U.S. application Ser. No. 16/521,466, filed on Jul. 24, 2019, now U.S. Pat. No. 11,179,249, which is a divisional of U.S. application Ser. No. 15/024,747, filed on Mar. 24, 2016, now U.S. Pat. No. 10,405,993, which is a national phase of PCT International Application No. PCT/IB2014/002711, filed on Nov. 11, 2014, which claims priority benefit under 35U.S.C. § 119 (c) to U.S. Provisional Application No. 61/903,814, filed on Nov. 13, 2013, both of which are incorporated by reference in their entirety herein. Any and all applications for which a foreign or domestic priority claim is identified in the Application Data Sheet as filed with the present application are hereby incorporated by reference under 37 CFR 1.57.

### BACKGROUND

### Field of the Invention

The present invention relates to shoulder implants. In particular, the present invention relates to glenoid implants for shoulder joints having scapula erosion and devices that facilitate implanting the same.

### Description of the Related Art

In a healthy shoulder joint, the head of the humerus interacts with the glenoid cavity of the scapula to form a “ball and socket” joint. The humeral head abuts and articulates with the glenoid to provide a wide range of motion. In an unhealthy shoulder joint, the interaction between the glenoid and the humerus is compromised, requiring repair or replacement.

Replacing the glenoid articular surface of the scapula of a human with a prosthetic glenoid component is a delicate surgical operation, notably because of the muscular environment of the shoulder. It is found that, depending on the position of implantation of such a glenoid component, risks of separation of the component from the underlying scapula exist due to forces applied to this component in subsequent movements of the prosthesized shoulder. In particular, in certain patients, it was found that, even if the implantation on their scapula of such a glenoid component was perfectly centered on the articular head of the corresponding humerus on completion of the surgical implantation operation, the resumption of their activities led, more or less rapidly, to instability of the prosthesis.

Currently, several companies are working on custom devices to guide glenoid bone preparation. One example filed by Tornier, Inc. is U.S. patent application Ser. No. 12/954,423, filed 24 Nov. 2010 and published as US 2011/0130795 A1.

### SUMMARY

A patient specific glenoid guide is provided to facilitate properly aligned implantation of a glenoid prosthesis into a patient. The guide shape is designed preoperatively based on the unique configuration of the scapula of the patient. The guide orientation is chosen preoperatively based on one or more of the bone structure of the patient, the wear pattern of the patient's glenoid cavity, the anchoring means of the glenoid prosthesis, or other aspects.

The guide may reversibly snap into securement with the scapula of the patient to hold the guide to the scapula during surgery. The guide may establish one or more axes through the shoulder joint about which subsequent bone preparation procedures and prosthesis implantation may be carried out. The guide may allow insertion of one or more K-wires or pins through the guide and into the scapula along the axis, and also allow removal of the guide without removing the one or more K-wires or pins.

The patient specific glenoid guide may be comprised of a central tubular element and three or more peripheral arms emanating from the central tubular element. One or more arms may terminate in a peripheral peg. One or more peripheral peg may be configured to reversibly engage with the scapula of the patient. The guide may be made by rapid prototyping or three dimensional printing methods.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates schematic views of worn shoulder joints.

FIG. 2 illustrates an isometric plan view of a patient specific glenoid guide.

FIG. 3 illustrates a partial cross sectional side view of a patient specific glenoid guide.

FIG. 4A illustrates a partial cross sectional side view of a patient specific glenoid guide.

FIG. 4B illustrates a cross sectional side view of a portion of a patient specific glenoid guide.

FIG. 5 illustrates an isometric plan view of a patient specific glenoid guide attached to a model of a patient's scapula.

FIG. 6 illustrates an isometric plan view of an alternate embodiment of a patient specific glenoid guide.

FIG. 7 illustrates an isometric bottom view of an alternate embodiment of a patient specific glenoid guide.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION OF THE PREFERRED EMBODIMENT

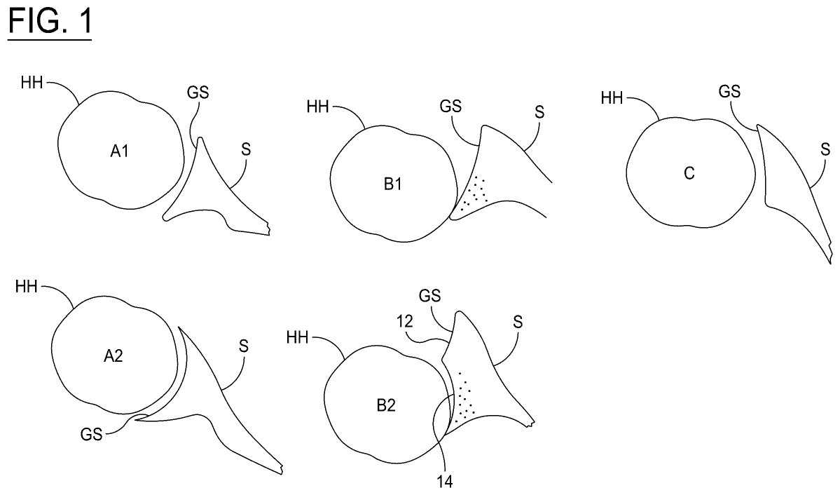

FIG. 1 illustrates five examples of worn shoulder joints that can be found in patients in need of shoulder arthroplasty. Each joint is comprised of a humeral head HH and a scapula S. The glenoid joint surface GS of the scapula is worn and can be classified as A1, A2, B1, B2, or C according to the shape of the glenoid surface (“Glenoid morphology in OA: Walch Classification”, Walch G. et. al., J. Arthroplasty, 14:756-760, 1999). A glenoid GS may include a neo-glenoid portion 14 that has a significant amount of erosion and a paleo-glenoid portion 12 that has little or no erosion. Such a glenoid is commonly referred to as a “type-B2” glenoid (J. Bone Joint Surg. Br. 2011 vol. 93-B no. SUPP IV, 571). A prosthetic glenoid component (not shown) can be adapted to be positioned between the scapula and the humeral component. The glenoid component is also adapted to articulate with the humeral component. The humeral component may be a humeral prosthesis secured to the humerus of the subject or an anatomical humeral head of the subject.

As part of the process for restoring a functional articular surface to the scapula a glenoid implant is firmly attached to the scapula by a fixation means (not shown). In some examples the fixation means comprises one or more screws, pegs, keels, fins, cement, or other fixation means. It is desirable to establish proper orientation of the glenoid implant in relation to the scapula and the humerus to assure that the fixation means has adequate strength to resist implant dislodgement from forces generated by articular motions of the joint. For example, screws must have adequate pull-out strength to resist articular forces of the joint that tend to dislodge the implant from the shoulder bones. Further, proper orientation of the glenoid implant in relation to the scapula and the humerus can minimize the forces generated on the glenoid implant during articulation of the shoulder joint. To facilitate proper orientation of the glenoid implant an axis through the shoulder joint can be established and the axis used to properly orient the glenoid implant so as to accomplish the above goals.

Another part of the process for properly fitting a glenoid implant to the scapula can be preparing the worn surface of the scapula so that the prepared surface will match a previously prepared surface of the glenoid implant. A surgeon may need to remove a significant amount of bone including cortical bone of the relatively healthy portions of the glenoid to accommodate typical glenoid implants. When these matched surfaces are brought into apposition the combination will resist rocking, sliding, twisting, and other articular motions of the joint that tend to dislodge the implant from the shoulder bones. To facilitate proper orientation of the glenoid implant an axis through the shoulder joint can be established and the axis used to guide scapula surface preparation tools such as reamers, guides, broaches and other devices so as to accomplish the above goals.

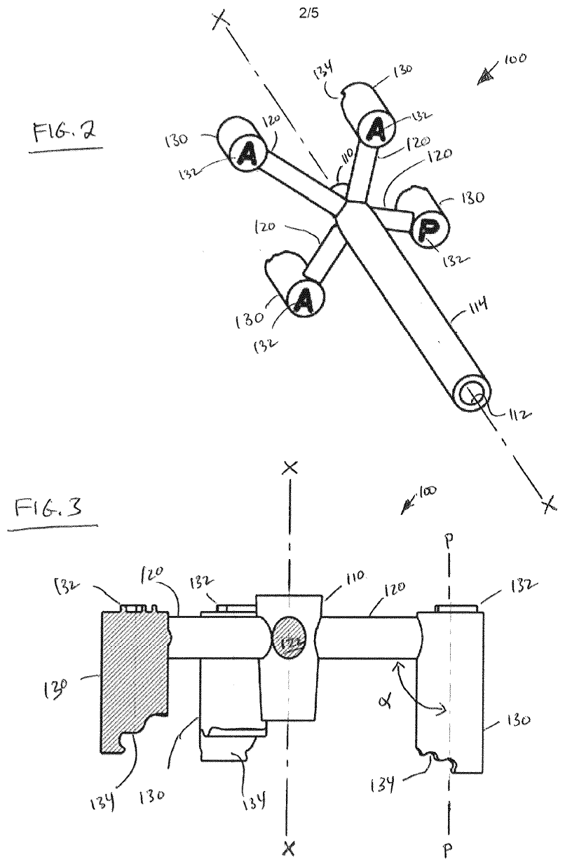

FIGS. 2 and 3 illustrate one embodiment of a patient specific glenoid guide. Guide 100 is a unique structure based on the exact shoulder joint anatomy of a specific patient. Guide 100 is comprised of central tubular element 110, three or more arms 120, and at least three arms each having peripheral peg 130.

Central tubular clement 110 is comprised of lumen 112 having axis X-X and is designed to guide a drill bit (not shown) for drilling a hole in the scapula. In another embodiment central tubular element 110 is designed to guide an alignment pin (not shown) through lumen 112 along axis X-X.

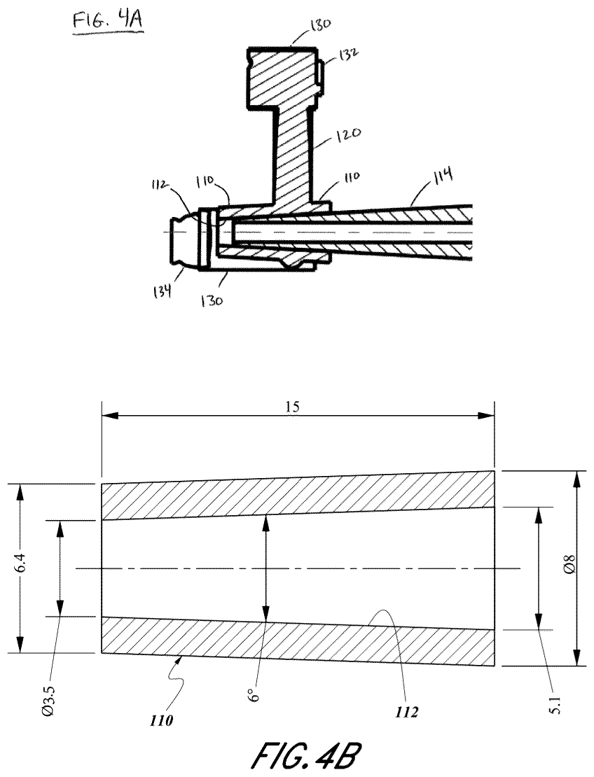

In a further embodiment (see FIG. 4A and 4B) guide 100 is used with commercially available metallic pin guide 114 of a generic design that is not patient specific. Pin guide 114 can be engaged with lumen 112 of central tubular clement 110 to hold the two components together during surgery. In one embodiment pin guide is engaged with central tubular clement by friction fit. In another embodiment pin guide is engaged with central tubular element by locking conical tapers, in one example Morse tapers. Specific dimensions (in mm) of central tubular element 110 for one locking taper embodiment are illustrated in FIG. 4B, and pin guide 114 external mating dimensions are the same as the internal mating dimensions of the central tubular element. Typically pin guide 114 is long enough to function as a handle during surgery for placement of guide 100.

At least three arms 120 are provided on the guide, four arms are preferred, and 5, 6, 7, or 8 arms are contemplated. The arms between the central tubular clement and each peripheral peg may have an elliptical, round, ovoid, polygonal, square, rectangular, triangular, other cross-section. In one embodiment the arm has an elliptical cross section and the major axis of the ellipse is perpendicular to the glenoid surface, the major axis is about 5 mm in length and the minor axis is about 4 mm in length. Arm 120 has a cross sectional area 122. Arm cross sectional areas of 10 square millimeters to 40 square millimeters and any cross sectional area therebetween are contemplated.

Peripheral peg 130 is comprised of identifier 132 and engagement surface 134. At least three pegs are provided on the guide, four pegs are preferred, and 5, 6, 7, or 8 pegs are contemplated. Three pegs are positioned to engage the anterior border of the glenoid cavity while one peg is positioned to engage the supero-posterior border of the glenoid cavity. Opposite to peg engagement surface 134, on the peg lateral extremity, the pegs are marked with identifier 132. In some embodiments anterior pegs are marked with identifier “A” while posterior pegs are marked with identifier “P”. Peg diameters of 6 mm to 10 mm and any diameter therebetween are contemplated. In one embodiment peg diameter is 8 mm. In another embodiment the posterior peg axis P-P is angled at an obtuse angle a from the arm so as to not be in conflict with the posterior retractor during the arthroplasty procedure. Engagement surface 134 may be customized to closely conform to the 3 dimensional shape of the border of the glenoid cavity.

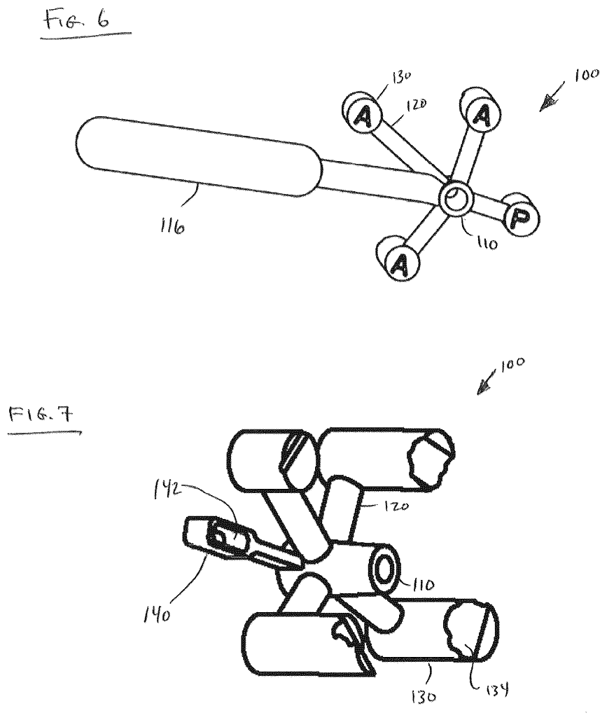

Patient specific glenoid guide 100 optionally comprises handle 116 (FIG. 6). Handle 116 is long enough to function as a handle during surgery for placement of guide 100. Also, use of handle 116 instead of pin guide 114 for manipulation of guide 100 allows a short drill bit to be used through central tubular clement 110 rather than a long drill bit through the combination of central tubular element 110 plus drill guide 114.

Patient specific glenoid guide 100 optionally comprises strut 140 having slot 142 (FIG. 7). The slot rotational position along the central axis X-X of the guide is the one of the supero-inferior axis of the glenoid component as determined by the pre-operative planning. Slot 142 is optionally used by the surgeon to mark this axis on the patient's bone with electrocautery.

In a further embodiment (not shown), second guide 100′ is associated with first guide 100. Second guide 100′ is similar to first guide 100 except that second guide 100′ is designed to fit the patient's scapula after the scapula shape has been modified, for example, modified by reaming, and central tubular element 110′ is comprised of two or more lumens 112′, 112″ having axes parallel to axis X-X. In some embodiment's lumens 112′, 112″ may be used to guide a drill bit (not shown) for drilling at least 2 holes in the scapula.

Guide 100 can be made by molding, machining, casting, thermal forming, or by other methods. In one embodiment guide 100 is made by rapid prototyping techniques, additive manufacturing or three dimensional printing using methods such as stereolithography or laser sintering. Guide 100 can be comprised of thermoplastics such as polyamide (such as PA2200 by Arptech) or metals such as titanium or stainless steel, or other materials.

Guide 100 is designed from three dimensional (3D) data about the anatomy of a patient's shoulder. The position and orientation of the axis X-X of the central tubular element 110 is defined according to pre-operative planning. The design process reproduces the translational positions of the glenoid component on three axes, (the antero-posterior axis, the supero-inferior axis and the medio-lateral axis) and it also reproduces the rotational positions of the glenoid component around the same three axes (supero-inferior axis (version), antero-posterior axis (inclination), and the medio-lateral axis (rotation)). By visualizing all of these positions a best choice position and orientation of axis X-X can be selected before operating on the patient. Also, a variety of glenoid implants can be evaluated for compatibility and performance in relation to the patients specific anatomy.

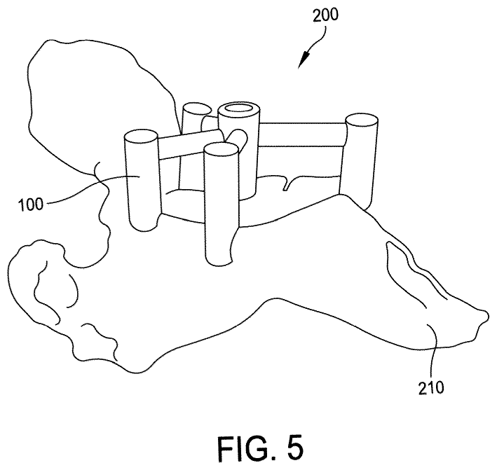

In one exemplar embodiment a three dimensional model of the patients shoulder is generated from imaging data obtained using a medical imaging technique such as a CT scan or a MRI. The imaging data are processed manually or automatically to generate a 3D model of the patient's scapula. From the 3D model of the patient's scapula, patient-specific guide 100 is designed automatically or manually using various CAD programs and/or software available such as Solid Works or ProEngineer.
a. As a first step in this example the 3D model of the bone is displayed in a specific environment in which the surgeon is able to select the desired implant and to place it at the desired location and desired orientation in the 3D model of the patient's scapula. Alternatively the implant could be automatically selected and/or placed based on a set of pre-determined criteria such as those described in EP2324801A1or US2011/0119884 A1 which are incorporated herein in their entirety and can be found in the Appendix.
b. As a second step in this example, once the implant is selected and placed in position in the 3D model of the patient's scapula the location of the pegs is determined by the surgeon or by an engineer. Then the guide structure, including central tubular clement 110, arms 120, pegs 130, and all of their features as described above, is manually or automatically generated. A dilatation of 2 pixels of the outside dimensions of the scapula is applied to bone model, and therefore to the guide engagement surface, in order to get a proper fit between the guide and the bone model as well as between the guide and the native bone.
c. The patient-specific guide 100 so generated will have a three dimensional engagement surface at the distal end of each peg that is complementary and made to conformingly contact the anatomical surface of the glenoid cavity border. The patient-specific guide 100 is thereby configured to fit at a unique position to the anatomical surface of the scapula. The central element of the guide so generated is in the proper location and orientation for proper location of the glenoid implant.
d. Optionally a 3D bone model 210 of the patient's scapula can be produced from the 3D imaging data, and said model can be provided to the surgeon with guide 100 as part of an implant kit 200 (FIG. 5). Such a model will allow the surgeon to test the fit and orientation of the guide 100 against the model of the patient's scapula prior to surgery, and can be a reference for the surgeon during surgery.

A non-limiting exemplar method of use of the patient specific glenoid guide is now described.
a. A patient undergoes a medical imaging technique such as a CT scan or a MRI and the imaging datum are processed manually or automatically to generate a 3D model of the patient's scapula.
b. The 3D model of the scapula is displayed in a specific environment in which the surgeon selects the desired implant and places it at the desired location and desired orientation in the 3D model of the patient's scapula. Alternatively the implant is automatically selected and/or placed based on a set of pre-determined criteria such as those described in EP2324801A1 or US2011/0119884 A1 which are incorporated herein in their entirety and can be found in the Appendix.
c. The location of 4 pegs is determined by the surgeon or by an engineer.
d. The guide structure, including central tubular element 110, arms 120, pegs 130, and all of their features as described above, is manually or automatically generated. A dilatation of 2 pixels of the outside dimensions of the scapula is applied to bone model, and therefore to the guide engagement surface, in order to get a proper fit between the guide and the bone model as well as between the guide and the native bone.
e. Optionally a 3D bone model of the patient's scapula is produced from the 3D imaging data.
f. The glenoid guide is provided to the surgeon. Optionally, a kit comprised of the 3D bone model and glenoid guide is provided to the surgeon.
g. The surgeon exposes the glenoid cavity of the patient.
h. The glenoid guide is pressed onto the border of the glenoid cavity and engages the glenoid in a snap fit attachment.
i. Pin placement through lumen 112 of guide 110 into patients scapula is performed.
j. Guide 110 is removed from the pin.
k. Scapula surface preparation and/or glenoid prosthesis placement is performed using pin as a guide.
l. Pin is removed.
m. Remainder of shoulder arthroplasty procedure is completed.

### Terminology

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments.

The term “about” as used herein represent an amount close to the stated amount that still performs a desired function or achieves a desired result. For example, the term “about” may refer to an amount that is within less than 10% of the stated amount, as the context may dictate.

Any methods disclosed herein need not be performed in the order recited. The methods disclosed herein include certain actions taken by a practitioner; however, they can also include any third-party instruction of those actions, either expressly or by implication. For example, actions such as “positioning three pegs around an anterior anatomical feature adjacent of the a glenoid cavity” include “instructing the positioning of three pegs around an anterior anatomical feature adjacent of the a glenoid cavity.”

Although certain embodiments and examples have been described herein, it will be understood by those skilled in the art that many aspects of the glenoid guide shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

Some embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

### EXAMPLE EMBODIMENTS

The following example embodiments identify some possible permutations of combinations of features disclosed herein, although other permutations of combinations of features are also possible.
1. A patient specific glenoid guide for attachment to a scapula of a patient, the glenoid guide comprising:
   a guide feature;
   at least three arms having a first end coupled with the guide feature and a second end disposed away from the guide feature; and
   at least three peripheral pegs, each peripheral peg extending from the second end of a corresponding arm and comprising an engagement surface;
   wherein the engagement surface is configured to engage the scapula of the patient to provide a snap fit attachment onto the scapula.
2. The patient specific glenoid guide of Embodiment 1, wherein the guide feature comprises a central tubular element that comprises a lumen.
3. The patient specific glenoid guide of Embodiment 1 or 2, wherein the guide feature is tapered.
4. The patient specific glenoid guide of any one of Embodiments 1 to 3, wherein the guide feature is configured to receive a pin guide.
5. The patient specific glenoid guide of any one of Embodiments 1 to 4, wherein the at least three peripheral pegs comprises four peripheral pegs.
6. The patient specific glenoid guide of Embodiment 5, wherein three of the four peripheral pegs are configured to engage an anterior anatomical feature of the scapula, and wherein one of the four peripheral pegs is configured to engage a supero-posterior anatomical feature of the scapula.
7. The patient specific glenoid guide of Embodiment 6, wherein three of the four peripheral pegs are configured to engage an anterior border of a glenoid cavity, and wherein one of the four peripheral pegs is configured to engage a supero-posterior border of the glenoid cavity.
8. The patient specific glenoid guide of any one of Embodiments 1 to 7, wherein each of the at least three arms comprises an elliptical cross section, the major axis of the elliptical cross-section being perpendicular to a glenoid cavity when the glenoid guide is coupled with the scapula.
9. The patient specific glenoid guide of any one of Embodiments 1 to 8, wherein an angle between one of the at least three arms and a corresponding peg is an obtuse angle.
10. The patient specific glenoid guide of any one of Embodiments 1 to 9, further comprising a lateral handle extending laterally from the guide feature.
11. The patient specific glenoid guide of any one of Embodiments 1 to 10, further comprising a strut extending laterally from the guide feature, the strut comprising a slot.
12. A system for guiding a glenoid prosthesis, the system comprising:
   the glenoid guide of any one of Embodiments 1 to 11; and
   a pin guide configured to engage the guide feature.
13. The system of Embodiment 12, wherein the pin guide comprises a tapered surface, and wherein the guide feature is tapered to receive the tapered pin guide.
14. A surgical kit comprising:
   a patient specific glenoid guide, the glenoid guide comprising:
   a guide feature;
   a plurality of arms extending from the guide feature; and
   a plurality of peripheral pegs, each of the plurality of peripheral pegs extending from a corresponding arm, each of the plurality of peripheral pegs comprising an engagement surface; and
   a three dimensional model of a patient's scapula comprising a glenoid cavity border; and
   wherein the engagement surfaces of the patient specific glenoid guide are configured to engage the glenoid cavity border of the model by a snap fit.
15. The surgical kit of Embodiment 14, wherein the guide feature comprises a central tubular element that comprises a lumen.
16. The surgical kit of Embodiment 14 or 15, wherein the guide feature is tapered.
17. The surgical kit of any one of Embodiments 14 to 16, wherein the guide feature is configured to receive a pin guide.
18. The surgical kit of any one of Embodiments 14 to 17, wherein the plurality of peripheral pegs comprises four peripheral pegs.
19. The surgical kit of Embodiment 18, wherein three of the four peripheral pegs are configured to engage an anterior portion of the glenoid cavity border, and wherein one of the four peripheral pegs is configured to engage a supero-posterior portion of the glenoid cavity border.
20. The surgical kit of any one of Embodiments 14 to 19, wherein each of the plurality of arms comprises an elliptical cross section, the major axis of the elliptical cross-section being perpendicular to a portion of the model corresponding to a glenoid cavity when the guide is applied to the model.
21. The surgical kit of any one of Embodiments 14 to 20, wherein an angle between one of the plurality of peripheral pegs and the corresponding arms is an obtuse angle.
22. The surgical kit of any one of Embodiments 14 to 21, further comprising a lateral handle extending laterally from the guide feature.
23. The surgical kit of any one of Embodiments 14 to 22, further comprising a strut extending laterally from the guide feature, the strut comprising a slot.
24. The surgical kit of any one of Embodiments 14 to 23, further comprising a pin guide coupled to the guide feature.
25. A method of guiding a glenoid prosthesis, the method comprising:
   pre-operatively determining a position and an orientation of guide feature of a glenoid guide based on a specific patient's scapula, the glenoid guide comprising:
   a plurality of arms extending from the guide feature; and
   a peripheral peg extending from each of the plurality of arms, the guide feature disposed inward of the peripheral pegs;
   engaging the glenoid guide with the scapula;
   inserting a pin having an axis through the guide feature to or through the scapula.
26. The method of Embodiment 25, wherein the guide feature comprises a central tubular element that comprises a lumen and inserting the pin comprises inserting the pin through the lumen of the guide feature.
27. The method of Embodiment 25 or 26, wherein engaging the glenoid guide with the scapula comprises engaging the glenoid guide with a glenoid cavity border by snap fit.
28. The method of any one of Embodiments 25 to 27, wherein engaging the glenoid guide with the scapula comprises positioning three pegs around an anterior anatomical feature of a glenoid cavity and positioning one peg at a supero-posterior anatomical feature of the glenoid cavity.
29. The method of Embodiment 28, wherein engaging the glenoid guide with the scapula comprises positioning three pegs around an anterior anatomical border of the glenoid cavity and positioning one peg at a supero-posterior border of the glenoid cavity.
30. The method of any one of Embodiments 25 to 29, further comprising advancing the glenoid prosthesis along the pin.
31. The method of any one of Embodiments 25 to 30, further comprising securing the pin to the guide feature by a friction fit.
32. The method of any one of Embodiments 25 to 30, further comprising securing a conical taper of the pin with a conical taper of the guide feature.

What is claimed is:

1. A system, comprising:
a patient-specific guide including:
a guide element defining a lumen that extends from a first side of the guide element to a second side of the guide element;
a plurality of arms, each arm of the plurality of arms having a first end coupled to the guide element and extending to a second arm such that each arm of the plurality of arms extends radially outward from the guide element; and
a plurality of peripheral pegs, each peripheral peg of the plurality of peripheral pegs coupled to the second end of a respective arm of the plurality of arms and extending from a lateral end to a medial end, the medial end including an engagement surface configured to conform to a three dimensional shape of a specific area of a border of a glenoid cavity of a scapula,
wherein, when the patient-specific guide is coupled to the scapula such that each engagement surface of each peripheral peg of the plurality of peripheral pegs engages the specific area of the border of the glenoid cavity, the plurality of arms are spaced apart from a lateral surface of the glenoid cavity.

2. The system of claim 1, further comprising:
a pin guide sized and configured to be received within a portion of the lumen, wherein the pin guide defines a central aperture; and
a pin sized and configured to be received within the central aperture defined by the pin guide.

3. The system of claim 2, wherein the pin guide is configured to be coupled to the portion of the lumen via a friction fit.

4. The system of claim 2, wherein pin guide is configured to be coupled to the portion of the lumen via locking conical tapers.

5. The system of claim 1, wherein the patient-specific guide includes a handle coupled to and extend away from the guide element.

6. The system of claim 5, wherein the patient-specific guide includes a strut having a first end coupled to the guide element, the strut eextending radially outward from the guide element at a location that corresponds to a supero-inferior axis of a glenoid prosthesis when the patient-specific guide is coupled to the scapula such that each engagement surface of each peripheral peg of the plurality of peripheral pegs engages the specific area of the border of the glenoid cavity.

7. The system of claim 6, wherein the strut defines a slot, the slot having a longitudinal axis that identifies the supero-inferior axis of the glenoid prosthesis when the patient-specific guide is coupled to the scapula such that each engagement surface of each peripheral peg of the plurality of peripheral pegs engages the specific area of the border of the glenoid cavity.

8. The system of claim 1, wherein:
the medial end of a first peripheral peg of the plurality of peripheral pegs is configured to engage the specific area of a super-posterior border of the glenoid cavity; and
the medial end of a second peripheral peg of the plurality of peripheral pegs is configured to engage of an anterior anatomical feature located at the specific area of the border of the glenoid cavity.

9. The system of claim 8, wherein each peripheral peg of the plurality of peripheral pegs is configured to engage the specific area of the border of the glenoid cavity with a snap fit.

10. The system of claim 1, wherein the lateral end of at least one peripheral peg of the plurality of peripheral pegs includes an identifier.

11. The system of claim 1, wherein the guide element has a cylindrical shape.

12. A kit, comprising:
a glenoid guide, including:
a guide element extending from a first medial side to a first lateral side, the guide element defining a lumen extending from the first medial side of the guide element to the second lateral side of the guide element;
a plurality of peripheral members arranged around the guide element, each peripheral member of the plurality of peripheral members extending from a second lateral side to a second medial side, the second medial of each peripheral member of the plurality of peripheral members including a patient-specific surface that is complementary to an area of a border of a glenoid cavity of a patient; and
a plurality of arms, each arm of the plurality of arms extending between and coupled to the guide element and a respective peripheral member of the plurality of peripheral members,
wherein each arm of the plurality of arms is spaced from a lateral surface of the glenoid cavity of the patient when the patient-specific surface of each of the plurality of peripheral members engages the area of the border of the glenoid cavity of the patient, and
wherein at least one peripheral member of the plurality of peripheral members is configured to engage a posterior border of the glenoid cavity of the patient.

13. The kit of claim 12, wherein the glenoid guide includes a positioning opening identifying a rotational position of the glenoid guide about an axis of the lumen.

14. The kit of claim 13, wherein the positioning opening is defined by a strut that extends from the guide element.

15. The kit of claim 13, further comprising:
a pin guide sized and configured to be received within a portion of the lumen, the pin guide defining a central aperture; and
a pin sized and configured to be received within the central aperture defined by the pin guide.

16. The kit of claim 15, wherein the glenoid guide is formed from a first material and the pin guide is formed from a second material that is different from the first material.

17. The kit of claim 16, wherein the pin guide is configured to be secured to the portion of the lumen via a tapered connection.

18. A method, comprising:
receiving medical image data for a specific patient;
designing a glenoid guide for the specific patient, the glenoid guide including:
a guide element extending from a first medial side to a first lateral side, the guide element defining a lumen extending from the first medial side of the guide element to the second lateral side of the guide element;
a plurality of peripheral members arranged around the guide element, each peripheral member of the plurality of peripheral members extending from a second lateral side to a second medial side, the second medial of each peripheral member of the plurality of peripheral members including a patient-specific surface that is complementary to an area of a border of a glenoid cavity of a patient; and
a plurality of arms, each arm of the plurality of arms extending between and coupled to the guide element and a respective peripheral member of the plurality of peripheral members,
wherein each arm of the plurality of arms is spaced from a lateral surface of the glenoid cavity of the patient when the patient-specific surface of each of the plurality of peripheral members engages the area of the border of the glenoid cavity of the patient, and
wherein at least one peripheral member of the plurality of peripheral members is configured to engage a posterior border of the glenoid cavity of the patient; and
manufacturing the glenoid guide.

19. The method of claim 18, wherein the medical image data is acquired from at least one of a CT scan and an MRI.

20. The method of claim 18, further comprising:
generating a three-dimensional model of a scapula of the specific patient from the medical image data; and
displaying the three-dimensional model of the scapula of the specific patient.
